(19) United States
(12) Patent Application Publication
(10) Pub. No.: US 2018/0161526 A1
(43) Pub. Date: Jun. 14, 2018

(54) SPRAY DEVICE FOR THE DELIVERY OF THERAPEUTIC AGENTS

(71) Applicant: TEAM CONSULTING LIMITED, Cambridge, Cambridgeshire (GB)

(72) Inventors: Philip David CANNER, Cambridge, Cambridgeshire (GB); David Stuart HARRIS, Cambridge, Cambridgeshire (GB)

(21) Appl. No.: 15/738,755

(22) PCT Filed: Jun. 23, 2016
(86) PCT No.: PCT/GB2016/051871

§ 371 Date: Dec. 21, 2017

(30) Foreign Application Priority Data
Jun. 24, 2015 (GB) 1511126.3

(51) Int. Cl. A61M 11/02; A61M 11/00

(52) U.S. Cl. CPC A61M 11/02; A61M 11/001; A61M 13/00; A61M 2206/20; A61M 2202/064

(57) ABSTRACT

A spray device (6) for the delivery of a therapeutic agent, the spray device (6) comprises a nozzle (1) having a first port (3) to input a gas source of positive gas pressure; a second port (4) to output gas from the gas source; and a Coanda surface (5) and a third port (2) between, and in fluid connection with, the first port (3) and the second port (4). The nozzle (1) is arranged such that, in use, a gas including a therapeutic agent enters the first port (3), the therapeutic agent having sufficient momentum to overcome forces from the gas flow over the Coanda surface (5) such that the therapeutic agent is ejected at the third port (2) in the form of a spray.

### FIELD OF THE INVENTION

The present invention relates to a spray device for the delivery of a therapeutic agent.

### BACKGROUND TO THE INVENTION

Management of bleeding during surgery is essential in ensuring successful patient outcomes. Bleeding is a major cause of death during trauma surgery, and post-operative bleeding occurs in approximately a quarter of all surgical procedures and is the most common surgical complication.

Haemostats, sealants and adhesives are considered by many to be established components of the surgeon's toolbox for managing bleeding, and are currently utilised as adjuncts to more traditional methods of achieving haemostasis such as sutures, mechanical clips and staples.

Haemostats, sealants and adhesives are a subset of many therapeutic agents that might be delivered to a wound or a surgical site. These agents may be in liquid, gel or powder form, and there are a number of methods of delivery, including but not limited to application of gauzes and foams carrying the agent, delivery of the agent via a syringe onto the site, and spraying in an aerosolised form.

Spraying has the benefit of enabling the surgeon to easily and evenly cover a wide area with the agent, or apply the agent to areas difficult to reach. The ability to easily cover a wide area is especially useful when attempting to control an “oozing” bleed from a large area, such as during liver resection, by applying a haemostatic agent; in this situation the use of mechanical clips and sutures is more difficult if not impossible.

Laparoscopic surgery, otherwise known as “keyhole” or “minimally invasive” surgery, is becoming increasingly common, primarily due to the shorter recovery times, less pain and bleeding, reduced scarring, and lower risk to the patient. During laparoscopic procedures the surgeon uses thin surgical tools inserted into the body through small incisions, typically into an inflated cavity. Haemostats, sealants and adhesives can be especially useful in laparoscopic surgery as the more traditional haemostatic techniques are more difficult to apply.

Spray devices for delivery of therapeutic agents are available in several forms. One form utilises a “hydraulic nozzle” principle: the device comprises a syringe connected to an atomising nozzle, and the application of force to the syringe plunger drives the agent through the atomising nozzle and generates a spray. In another form, frequently termed “gas assisted”, the liquid agent is introduced into a high-speed stream of gas, thus generating an aerosol. The gas assisted approach has advantages over the hydraulic nozzle approach, including the production of finer droplets and a lower dependence on the flow rate and hence the user to produce fine droplets.

Haemostats are also available in the form of a dry powder, which has advantages over the liquid form including greater stability at room temperature. For delivering powdered haemostats and other powdered therapeutic agents, the powder is typically introduced into a moving stream of gas. This stream of gas carries the powder and exits the device with the powder. If the device is held close enough to the tissue, the jet of gas will impact the tissue which in the worst case can lead to gas embolism and potential death.

To date, there have been six life threatening or fatal events caused by gas embolism relating to the use of spray devices, caused by the use of the devices at above recommended pressures and/or in too close proximity to the tissue. While there are device design strategies that can be used to limit the gas pressure, proximity is user controlled and can be more difficult to judge, especially in laparoscopic surgery.

### BRIEF SUMMARY OF THE INVENTION

It is therefore the object of the invention to provide a means of delivering therapeutic agents in the form of a spray without a flow of gas exiting the spray nozzle, thus reducing or removing the risk of gas embolism caused by using the device in too close proximity to the tissue.

Arrangements are described in more detail below and take the form of a spray device for the delivery of a therapeutic agent, the spray device comprises a nozzle having a first port to input a gas source of positive gas pressure; a second port to output gas from the gas source; and a Coanda surface and a third port between, and in fluid connection with, the first port and the second port. The nozzle is arranged such that, in use, a gas including a therapeutic agent enters the first port, the therapeutic agent having sufficient momentum to overcome forces from the gas flow over the Coanda surface such that the therapeutic agent is ejected at the third port in the form of a spray.

The invention is defined in the independent claims below to which reference should now be made. Preferred features are set out in the dependent claims.

In an aspect of the present invention, there is provided a spray device for the delivery of a therapeutic agent, the spray device comprising: a nozzle having: a first port to input a gas source of positive gas pressure; a second port to output gas from the gas source; and a Coanda surface and a third port between, and in fluid connection with, the first port and the second port; the nozzle being arranged such that, in use, a gas including a therapeutic agent enters the first port, the therapeutic agent having sufficient inertia to overcome forces from the gas flow over the Coanda surface such that the therapeutic agent is ejected at the third port in the form of a spray.

The therapeutic agent may comprise a powder or a liquid.

In another aspect of the present invention, there is provided a spray device for the delivery of a therapeutic agent, the spray device comprising: a nozzle having: a first port to input a gas source of positive gas pressure a second port to output gas from the gas source; and a Coanda surface and a third port between, and in fluid connection with, the first port and the second port; the nozzle being arranged such that, in use, a gas including a therapeutic agent enters the first port, the therapeutic agent having sufficient momentum to overcome forces from the gas flow over the Coanda surface such that the therapeutic agent is ejected at the third port in the form of a spray.

The therapeutic agent may comprise a powder or a liquid. The second port may be connected to a vacuum source. The vacuum from the vacuum source and the Coanda surface may prevent the gas from exiting the nozzle through the third port. The first port may have an inner sidewall that extends along an axis of the nozzle. The spray device may further comprise a projection projecting from the second port perpendicular to the inner sidewall. The projection may be spaced from and extend around the Coanda surface. The projection may terminate in a tip. The tip may be spaced from the sidewall in a direction perpendicular to the axis of the nozzle. The cross sectional area of the first port may reduce between an entrance of the first port and the Coanda surface to form an acceleration region. The acceleration region may have a constant cross sectional area along its length. The acceleration region may have a length that determines residence time of the therapeutic agent within the acceleration region and the subsequent exit velocity of the therapeutic agent. The device may contain an internal pressure and vacuum source. The spray device may be a laparoscopic powder delivery device.

A nozzle for the spray device describe above may be provided.

In a yet further aspect of the present invention, there is provided a method for the delivery of a therapeutic agent from a spray device, the method comprising: a gas including a therapeutic agent flowing into a first port of a nozzle of the spray device and being output from a second port of the nozzle, the therapeutic agent having sufficient momentum to overcome forces from the gas flow over a Coanda surface of the nozzle between, and in fluid connection with, the first port and the second port; such that the therapeutic agent is ejected at a third port of the nozzle, between, and in fluid connection with, the first port and the second port, in the form of a spray.

### BRIEF DESCRIPTION OF THE DRAWINGS

The invention will be described in more detail, by way of example, with reference to the accompanying drawings, in which:

FIGS. 1 is a schematic view of a nozzle embodying an aspect of the present invention showing the internal structure with dashed lines;

FIG. 2 is an end view of the nozzle of FIG. 1;

FIG. 3 is a cross section of the nozzle of FIGS. 1 and 2 through line A-A of FIG. 2;

FIG. 4 is the cross section of FIG. 3 illustrating gas flow through the nozzle;

FIG. 5 is the cross section of FIG. 3 illustrating gas and particular or droplet flow through the nozzle;

FIG. 6 is a side view of the nozzle of FIGS. 1 to 5 as part of a laparoscopic powder delivery device;

FIG. 7 is a perspective view of the laparoscopic powder delivery device of FIG. 6;

FIG. 8 is a side view of the nozzle of FIGS. 1 to 5 of a laparoscopic powder delivery device with an internal pressure and vacuum source;

FIG. 9 is a graph of particle velocity as a function of distance travelled along the acceleration region for a range of particle diameters of the nozzle of FIGS. 1 to 5;

FIG. 10 is a graph of particle momentum as a function of distance travelled along the acceleration region for a range of particle diameters of the nozzle of FIGS. 1 to 5;

FIG. 11 is a schematic generated by a computational fluid dynamics simulation of gas flow velocity vectors around the Coanda surface of the nozzle of FIGS. 1 to 5;

FIG. 12 is a schematic of contours of velocity magnitude in m/s generated by the computational fluid dynamics simulation of FIG. 11; and

FIG. 13 is a schematic generated by a computational fluid dynamics simulation of accelerated 50 μm diameter particles overcoming aerodynamic drag forces from gas flow around the Coanda surface of the nozzle of FIGS. 1 to 5.

In the drawings and text, like features have been given like reference numerals.

### DETAILED DESCRIPTION OF THE INVENTION

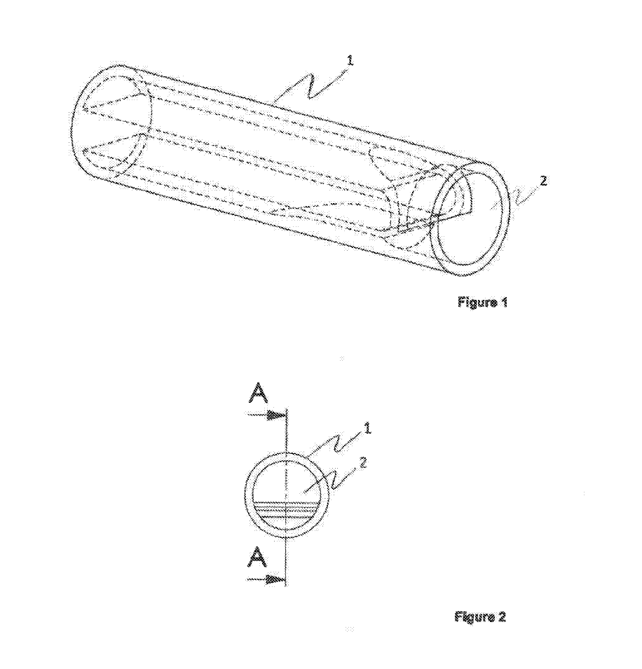

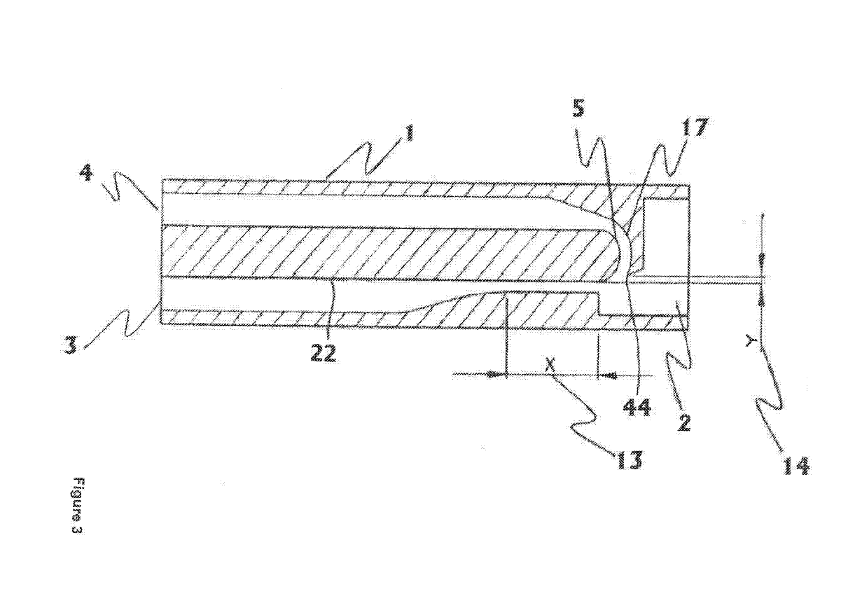

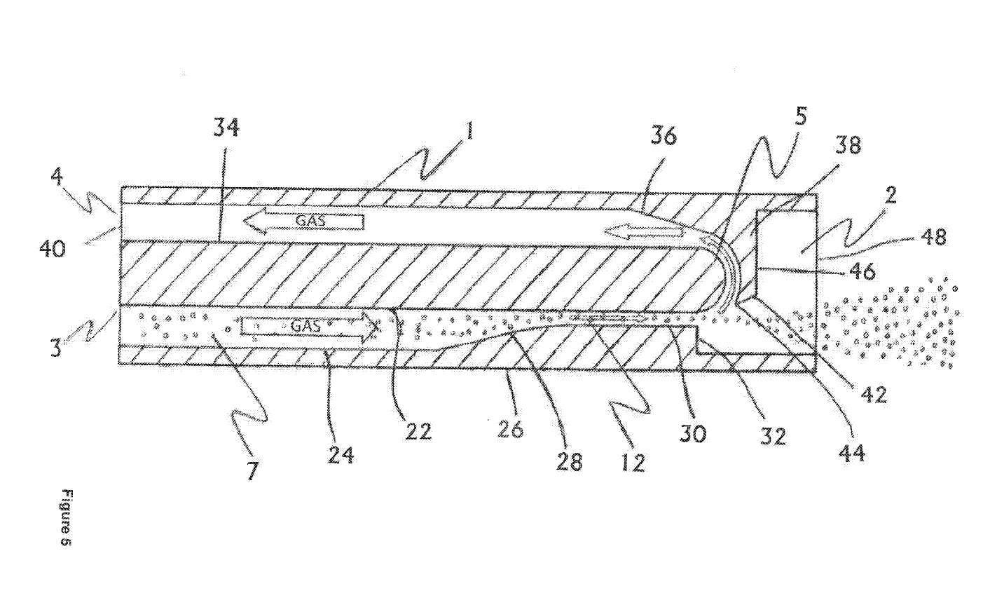

Embodiments of a spray device for the delivery of a therapeutic agent will now be described with reference to FIGS. 1 to 13. The spray device is a 5 mm diameter laparoscopic surgery tool that has a 5 mm diameter tip formed by a nozzle of a type described in detail below.

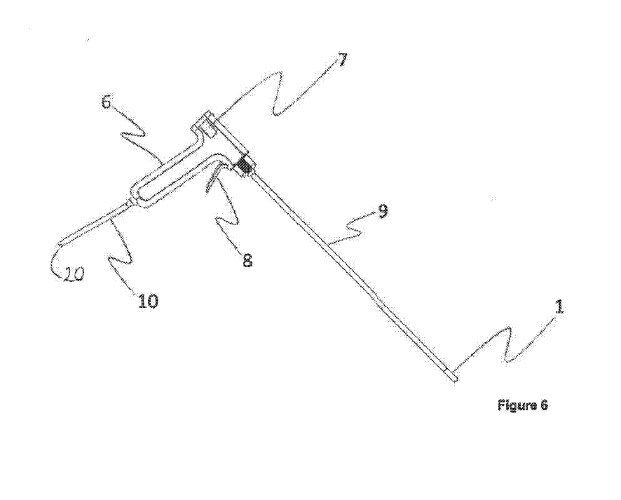

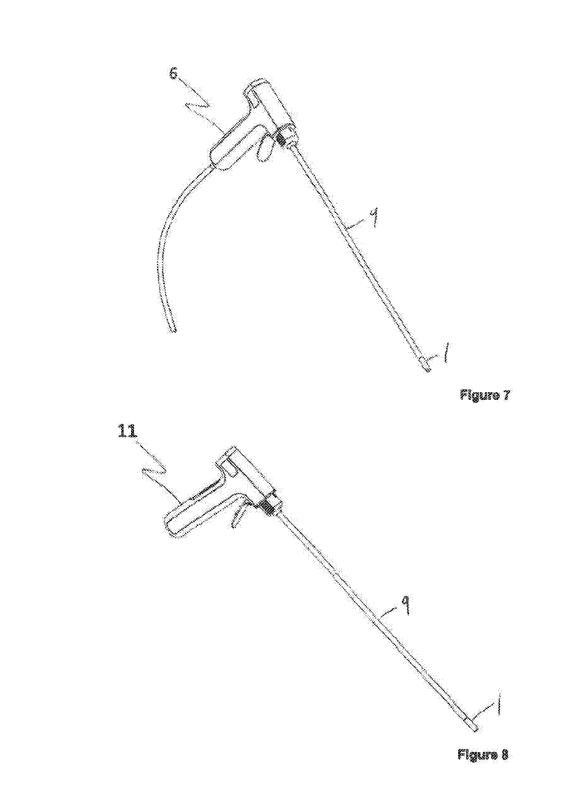

In one embodiment, the device 6 delivers a powder. The therapeutic agent is included in the powder. Such a device is illustrated in FIGS. 6 and 7, which illustrates a laparoscopic powder delivery device. It include a supply tube 20 that provides a flow or stream of gas into the device and a vacuum source to suck or pull gas from the device. The device includes a vial of powder 7 and a control button or trigger 8 for controlling the flow of powder into the stream of gas travelling along a first tube 10 within the supply tube into an outer or delivery tube 9 and to a nozzle 1 where the therapeutic agent exits or is output from the device. The tubes may be totally or partially rigid.

The device 6 is connected at one end (at the supply tube 20) to a source of positive gas pressure and is connected at the other end to the nozzle 1. The device also includes a means for introducing the powder into the flow of gas within the first tube, the flow of gas carrying the powder along the first tube to the nozzle 1. The device also includes a second tube 9 connected to the nozzle 1 and connected at its other end (at the supply tube 20) to a vacuum source (not illustrated), the second tube acting as a return line for the gas.

The nozzle 1 is illustrated in detail in FIGS. 1 to 5 and we now refer, in particular, to these Figures. In this example, the diameter of the nozzle 1 is 5 mm.

Referring in particular to FIG. 5, the nozzle 1 has three ports—the first port 3 is connected to the first tube 10, the second port 4 is connected to the second tube , and the third port 2 is open—it is out of this third port that the powder is sprayed. The ports are all in fluid connection with each other within the nozzle 1.

Within the nozzle 1 there is a curved Coanda surface 5 between the first and second ports.

A Coanda surface 5 is a surface that causes the Coanda effect. The Coanda effect is the tendency of a fluid jet to be attracted to a nearby surface.

Externally, the nozzle 1 is a circular cylinder that extends along an axis. The first port 3 extends internally with a constant cross sectional area from one end along the axis of the nozzle and narrows with reduced cross sectional area towards the other end forming an acceleration region 12 of the same cross sectional area throughout. A sidewall 22 forming an inner sidewall of the first port closest to the centre of the nozzle is straight extending parallel to or along the axis of the nozzle and then curves at its end forming the Coanda surface 5. This inner sidewall curves with a semi-circular cross section that forms the end of the second port 4. A sidewall forming an outer sidewall 24 of the first port closest to the outer surface 26 of the nozzle extends straight and parallel to the inner sidewall then slopes inwardly 28 towards the inner sidewall before continuing straight and parallel 30 to the inner sidewall and the axis of the nozzle. This straight portion forms the acceleration region 12. Axially, where the inner sidewall starts to curve, the outer sidewall has a step 32 outwardly, perpendicular to the axis of the nozzle. The second port has an inner sidewall 34 that extends straight, from a curved end that forms the Coanda surface, parallel to the axis of the nozzle. The second port has an outer sidewall 36 that curves with the same curvature as the Coanda surface (this forms a projection 38 of the second port outer sidewall; the projection is spaced from and extends around the Coanda surface) and then slopes outwardly or widens away from the curved end before extending straight and parallel to the inner sidewall to the exit 40 connected to the second tube. In this way, the cross sectional area of the first port reduces between an entrance of the first port and the Coanda surface to form the acceleration region. The acceleration region has a constant cross sectional area along its length. The acceleration region has a length that determines residence time of the therapeutic agent within the acceleration region and the subsequent exit velocity of the therapeutic agent. This is discussed in more detail further below.

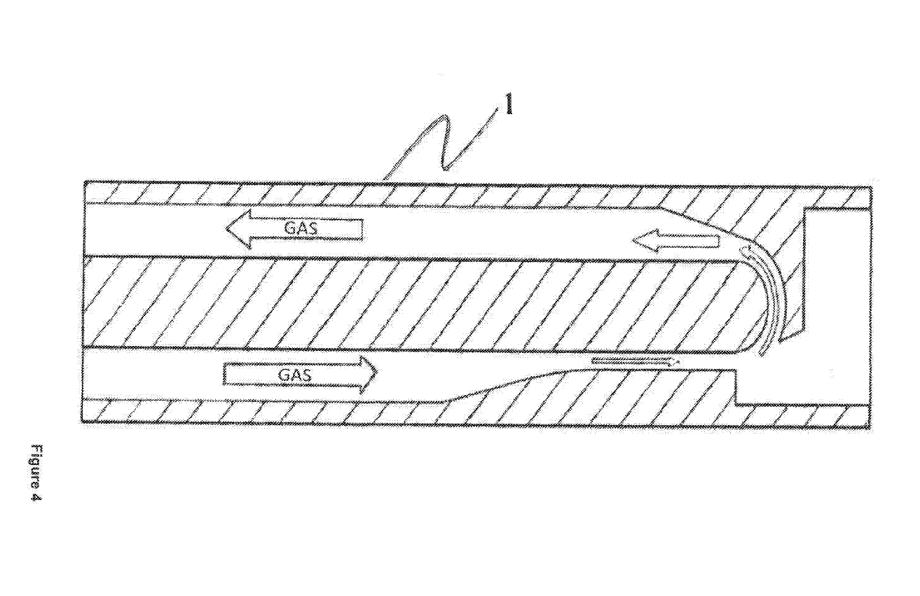

The third port 2 is formed by a slope 42 of the projection 38 of the second port outer sidewall 36 away from the Coanda surface 5 from a tip 44 to a step 46 perpendicular to the axis of the nozzle 1; the projection projects from the second port perpendicular to the inner sidewall and terminates in a tip. This forms a wide exit 48 of the third port 2. As illustrated best in FIG. 3, there is a vertical offset or space 14 of length Y between the tip 44 and the straight portion of the inner sidewall 22 of the first port 3 closest to the centre of the nozzle. This vertical offset or space is a significant dimension and it is discuss in more detail below.

As illustrated best in FIG. 5, the principal of operation is that the gas travels down the first tube 10 and enters the nozzle's first port 3. Upon reaching the Coanda surface 5, the gas follows the curve of the Coanda surface 18 and exits the nozzle 1 through the second port 4 and travels along the second tube towards the vacuum source. The Coanda effect and the vacuum prevent the gas from exiting the nozzle through the third port 2. The powder particles 7 (including therapeutic agent) are accelerated by the gas stream and travel with the gas down the first tube and enter the first port 3. In our design, the particles are accelerated within the acceleration region 12 within the nozzle to have sufficient momentum to overcome the aerodynamic drag forces acting upon them by the gas flow following the Coanda surface, and are subsequently ejected 19 through the third port 2. The geometry of the acceleration region 12 and the gas flow rate through the acceleration region are key design parameters that can be adjusted in order to optimise nozzle performance. The cross-sectional area for the gas to flow through of the acceleration region 12 and the gas flow rate determine the mean gas velocity for accelerating powder particles. The length of the acceleration region 13 determines the residence time of particles within the acceleration region 12 and the subsequent exit velocity of particles 15.

The exit momentum of particles from the acceleration region 12 varies significantly with particle diameter 16. The design parameters for the acceleration region such as the region length 13 can be adjusted in order to ensure that powder particles of a known size distribution exit the region with sufficient momentum to overcome the aerodynamic drag forces acting upon them by the gas flow following the Coanda surface 5, and are subsequently ejected 19 through the third port 2. The gas flow 18 is intended to be isokinetic (constant speed) as it flows around the Coanda surface 5. The vertical offset 14 is included between the exit of the acceleration region 12 and the beginning of the outer wall 17 that defines the flow path to the second port 4. This offset 14 is a key design feature for nozzle performance. Decreasing the vertical offset 14 can improve the effectiveness of the Coanda surface for directing the gas flow towards the second port 4. Increasing the vertical offset 14 can increase the proportion of particles that avoid collision with the outer wall 17 and are successfully ejected from the third port 2 rather than returning with the gas flow to the second port 4.

The magnitude of the vertical offset 14 can be optimised to achieve a balance between minimising the return of particles of a known size distribution to the second port 4 and maximising the effectiveness of the Coanda surface 5 for directing the gas flow towards the second port (4).

In other words, advantageously, the particular shape and configuration of the internal features of the nozzle provide for effective control of the nozzle.

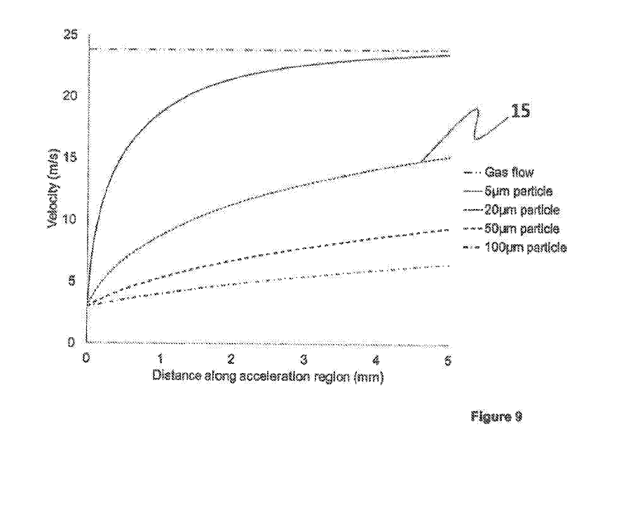

FIG. 9 is a graph of particle velocity as a function of distance travelled along the acceleration region 12 for a range of particle diameters (5 μm, 20 μm, 50 μm and 100 μm) of the nozzle 1 of FIGS. 1 to 5. As shown in FIG. 9, the smaller the particle size the higher the velocity. The velocity of the smallest particle size approaches the velocity of the gas flow towards the end of the acceleration region. The velocity of the larger particle sizes is some way from that of the velocity of the gas flow even towards the end of the acceleration region.

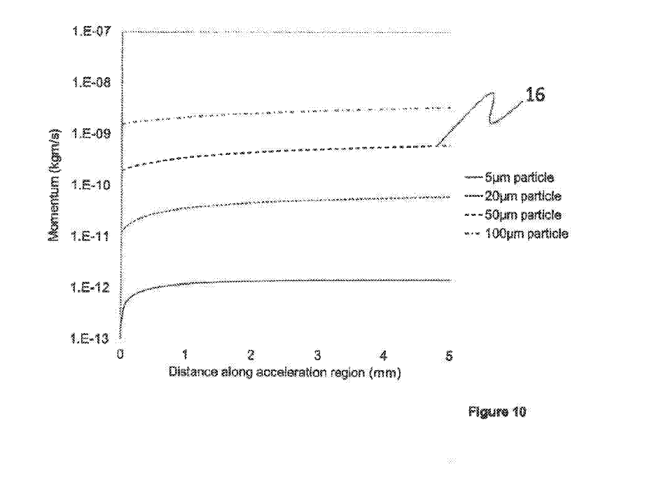

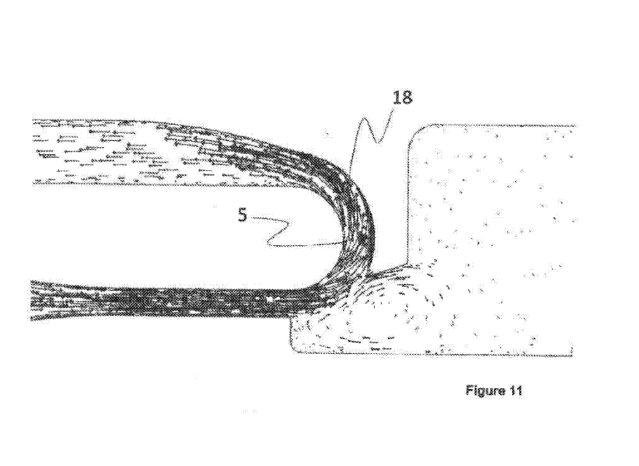

FIG. 10 is a graph of particle momentum as a function of distance travelled along the acceleration region for the same range of particle diameters of FIG. 9 for the same nozzle. The momentum of the particles change little once they have travelled a short distance into the nozzle. Larger particles have a higher momentum than smaller particles.

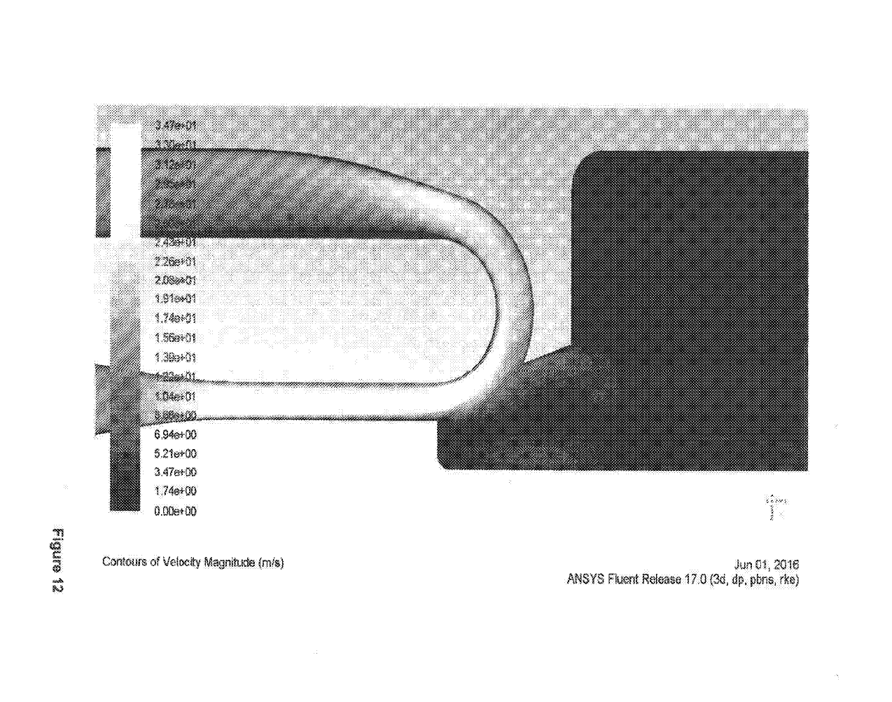

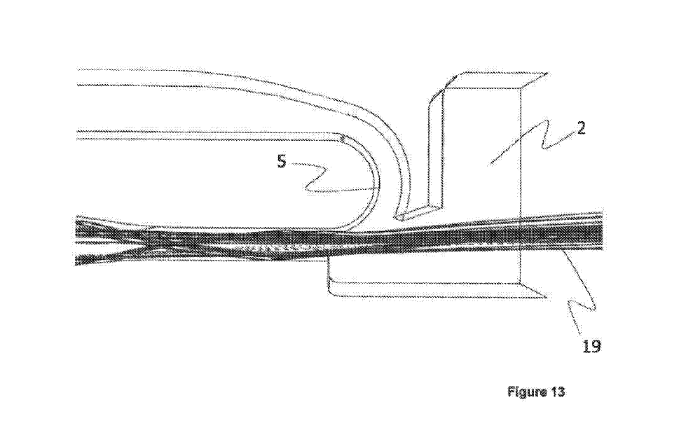

FIG. 11 is a schematic generated by a computational fluid dynamics simulation of gas flow velocity vectors around the Coanda surface of the nozzle of FIGS. 1 to 5. FIG. 12 is a schematic of contours of velocity magnitude in m/s generated by the computational fluid dynamics simulation of FIG. 11. In both of FIGS. 11 and 12, gas is shown clearly flowing around the Coanda surface with little exiting the nozzle. FIG. 13 is a schematic generated by a computational fluid dynamics simulation of accelerated 50 μm diameter particles overcoming aerodynamic drag forces from gas flow around the Coanda surface of the nozzle of FIGS. 1 to 5. These particles are shown exiting the nozzle. None continue around the Coanda surface. There is no turbulence.

The use of a Coanda surface to divert a stream of gas away from its direction of flow while not substantially affecting the direction of particles carried in the gas stream is described in U.S. Pat. No. 5,525,510, which does not feature a vacuum drawing air away from the nozzle. The addition of a vacuum allows the radius of curvature of the Coanda surface to be substantially smaller, thus allowing it to be made small enough to be used in the tip of a 5 mm diameter laparoscopic surgery tool. Without the vacuum, the minimum radius of curvature of the Coanda surface would be substantially larger to prevent the gas breaking away from the surface, and would be too large to be usable as a tool for use in laparoscopic surgery. The use of a vacuum to create a volumetric flow-rate of gas away from the nozzle equal to or greater than the volumetric flow-rate of gas towards the nozzle also guarantees zero net gas flow out of the end of the nozzle.

The use of a vacuum to divert a high speed flow of gas away from its direction of flow while the momentum of the particles causes them to continue in an axial direction is described in U.S. Pat. No. 7,892,836 B2, which does not utilise the Coanda effect. The patent describes a first gas tube carrying gas and particles to the nozzle tip, this first tube being surrounded by a second gas tube carrying gas away from the nozzle tip, the second gas tube extending further than and being substantially co-axial to the first gas tube. The disadvantage of not using the Coanda surface is that the flow is likely to be turbulent at the nozzle, which may allow occasional jets of gas to not be diverted and to leave the nozzle in the axial direction. Persons skilled in the art may assume that the addition of a Coanda surface at the end of the first gas tube may enhance the performance. However, we have found that this approach does not work with an axisymmetric co-axial arrangement, as the gas flow emanating from the first tube would be forced to split outwards apart from itself, making it unlikely to adhere to the Coanda surface in a stable manner and resulting in an unstable and turbulent gas flow.

The use of a Coanda surface creates a more controlled and controllable gas flow-path, and could allow the volumetric flow-rate of gas towards the nozzle to be equal and opposite to the volumetric flow-rate of gas being drawn away from the nozzle by the vacuum, thus making a fully recirculating gas-flow system possible. The use of a Coanda surface also enables easier tailoring of the gas and particle-flow.

In one arrangement, the flow of gas may be equal and opposite in direction in the first and second tubes. In this way there could be no net flow of gas out of the third port 2. In this arrangement, the first and second tubes could be connected, and the flow of gas generated by a peristaltic pump acting on the tube or a fan within the flow path, for example.

In another arrangement, the flow of gas away from the nozzle along the second tube is greater than the flow of gas towards the nozzle in the first tube. This will create a net flow of gas through the third port 2 back into the nozzle.

In another embodiment, the device delivers a liquid spray. In one arrangement, the liquid may be atomised and then introduced into the flow of gas in the first tube. In another arrangement, the liquid may be introduced as a liquid (rather than as a mist) into the flow of gas in the first tube, the flow of gas atomising the liquid (i.e. “gas assisted” atomisation). In either of these arrangements, the principle of operation is similar to the powder delivery device, in that the gas carrying the liquid droplets 7 follows the Coanda surface and does not exit the nozzle through the third port 2. The droplets have sufficient momentum to overcome the aerodynamic drag forces acting upon them by the gas flow following the Coanda surface, and are subsequently ejected through the third port 2 19.

In another arrangement, two or more liquids or powders may be introduced into the first tube.

In another arrangement, there may be multiple inlets to the nozzle, and the nozzle may have multiple Coanda surfaces, to allow for different liquids or powders to be sprayed without mixing within the nozzle.

In any of the arrangements, the flow of gas through the first and second tubes may be constant, and the powder or liquid added only when it is desired to be sprayed. The rate of powder or liquid addition may be controlled by the operator.

In one embodiment, the device is a laparoscopic powder delivery device 6. It may feature a vial of powder 7, and a control button 8 for controlling the flow of powder into the stream of gas travelling down a first tube to the nozzle 1. Both first tube and second tube may run within or be formed by an outer tube 9 which may be totally or partially rigid. Both first tube and second tube may run within or be formed by an outer tube 10 which may be flexible, and runs to the gas pressure source and vacuum pressure source.

In another embodiment, the device is a laparoscopic liquid spray delivery device 6.

In another embodiment, illustrated in FIG. 8, the device 11 is the same as the device of FIGS. 6 and 7, except that it contains an internal pressure and vacuum source (the gas pressure source and the vacuum source are integral with the device), and there is no need for the first and second tubes to run to an external pressure and vacuum source. This internal pressure and vacuum source may be an electronic pump, fan or impeller powered by a battery, or a miniature compressed gas cylinder with vacuum generator.

The invention could equally be embodied in a device more suitable for open surgery, for example it could be substantially as the devices 6 of FIG. 6 and 7 or 11 of FIG. 8 but with a shorter tube 9.

Embodiments of the present invention have been described. It will be appreciated that variations and modifications may be made to the described embodiments within the scope of the present invention.

1. A spray device for the delivery of a therapeutic agent, the spray device comprising:
a nozzle having:
a first port to input a gas source of positive gas pressure
a second port to output gas from the gas source; and
a Coanda surface and a third port between, and in fluid connection with, the first port and the second port;
the nozzle being arranged such that, in use, a gas including a therapeutic agent enters the first port, the therapeutic agent having sufficient momentum to overcome forces from the gas flow over the Coanda surface such that the therapeutic agent is ejected at the third port in the form of a spray.

2. A spray device according to claim 1, wherein the therapeutic agent comprises a powder or a liquid.

3. A spray device according to claim 1, wherein the second port is connected to a vacuum source.

4. A spray device according to claim 3, wherein the vacuum from the vacuum source and the Coanda surface prevent the gas from exiting the nozzle through the third port.

5. A spray device according to claim 1, wherein the first port has an inner sidewall that extends along an axis of the nozzle.

6. A spray device according to claim 5, further comprising a projection projecting from the second port perpendicular to the inner sidewall.

7. A spray device according to claim 6, wherein the projection is spaced from and extends around the Coanda surface.

8. A spray device according to claim 6, wherein the projection terminates in a tip.

9. A spray device according to claim 8, wherein the tip is spaced from the sidewall in a direction perpendicular to the axis of the nozzle.

10. A spray device according to claim 1, wherein the cross sectional area of the first port reduces between an entrance of the first port and the Coanda surface to form an acceleration region.

11. A spray device according to claim 10, wherein the acceleration region has a constant cross sectional area along its length.

12. A spray device according to claim 11, wherein the acceleration region has a length that determines residence time of the therapeutic agent within the acceleration region and the subsequent exit velocity of the therapeutic agent.

13. A spray device according to claim 1, wherein the device contains an internal pressure and vacuum source.

14. A spray device according to claim 1, wherein the spray device is a laparoscopic powder delivery device.

15. A nozzle for the spray device according to any preceding claim 1.

16. A method for the delivery of a therapeutic agent from a spray device, the method comprising: a gas including a therapeutic agent flowing into a first port of a nozzle of the spray device and being output from a second port of the nozzle, the therapeutic agent having sufficient momentum to overcome forces from the gas flow over a Coanda surface of the nozzle between, and in fluid connection with, the first port and the second port;
such that the therapeutic agent is ejected at a third port of the nozzle, between, and in fluid connection with, the first port and the second port, in the form of a spray.
